(19) United States
(12) Patent Application Publication
(10) Pub. No.: US 2006/0091015 A1
(43) Pub. Date: May 4, 2006

(54) Surface modification for non-specific adsorption of biological material

(75) Inventor: Aldrich N.K. Lau, Palo Alto, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 10/979,645

(22) Filed: Nov. 1, 2004

(51) Int. Cl. C07K 1/26; B03C 5/02

(52) U.S. Cl. 204/643; 204/450

(57) ABSTRACT

The present teachings provide a manipulation chamber, device, and method related to surface modifiers added to an electrode exposed to the biomolecules or a layer adjacent to an electrode exposed to the biomolecules to decrease non-specific adsorption of the biomolecules such as proteins or nucleic acids in a biological sample.

### FIELD

The present teachings relate to devices and methods for manipulation of small and microscopic objects such as cells or nucleic acids.

### BACKGROUND

Dielectrophoresis (DEP) is the analog of optical tweezers that are capable of manipulating objects, cells, and even a single molecule in an aqueous solution (P. J. Burke, Nano-dielectrophoresis: Electronic nanotweezers, 2003, Encyclopedia of Nanoscience and Nanotechnology, American Scientific). DEP refers to the lateral motion imparted on uncharged objects as a result of polarization induced by non-uniform electric fields (H. A. Pohl, Dielectrophoresis, Cambridge University Press, 1978). An analytical expression of DEP force is illustrated in FIG. 3 (T. B. Jones, Electromechanics of Particles, Cambridge University Press, 1995), where υ is the volume of the object, the factor in parentheses is the RMS value of the electric field, and αᵣ is the real part of the Clausius-Mosotti factor which relates the dielectric constant of the object εₚ and dielectric constant of the medium εₘ The star (*) denotes that the dielectric constant is a complex quantity. The term can have any value between 1 and −½, depending on the applied AC frequency and the dielectric constants of the object and medium. If is less than zero, it is called a negative dielectrophoresis in which the particle is capable of moving towards a lower electric field.

If the particles are charged, then electrophoresis (EP) occurs, instead of DEP, under DC current or low frequency AC. EP refers to the lateral motion imparted on charged objects in a non-uniform or uniform electric field.

DEP has been used to manipulate objects (N. G. Green, et al., J. Phys. D., 1997, 30, 2626-2633), to separate viable/non-viable yeast (G. H. Markx, et al., J. Biotechnology, 1994, 32, 29-37) and other micro-organisms such as separating Gram-positive bacteria from Gram-negative bacteria (G. H. Marks, et al., Microbiology, 1994, 140, 585-591), and to remove human leukemia cells and other cancer cells from blood (F. F. Becker, et al., J. Phys. D.: Appl. Phys., 1994, 27, 2659-2662; F. F. Becker, et al., Proc. Nat. Acad. Sci. (USA), 1995, 92, 860-864). The cells are manipulated by a traveling wave generated by a series of patterned electrodes lining up and charged with phase-shifted AC signals (A. D. Goater, et al., J. Phys. D., 1997, 30, L65-L69). The patterned electrodes can be patterned in an independently controlled array to provide such a traveling wave.

Optically activated DEP systems have been compiled using low-power laser light focused to induce DEP between two pattern-less surfaces, such as a indium tin oxide (ITO) transparent glass electrode and a substrate coated with photoconductive material to complete the circuit (P. Y. Chiou, et al., Cell Addressing and Trapping using Novel Optoelectric Tweezers, 2004, IEEE International Conference on Micro Electro Mechanical Systems, Technical Digest, 17^{th} Maastricht, Netherlands, Jan. 25-29, 2004). A non-uniform field is created by a well-defined laser spot and the objects in the liquid layer in between the two electrodes are polarized and move away from the illuminated spot by the negative or positive dielectrophoretic force. Silicon nitride coats the photoconductive material to provide separation between the photoconductive material and the liquid layer. Typical light activated DEP relies on a transparent ITO electrode to permit a focused laser beam to pass through the ITO electrode and illuminate a photoconductor. If the transparent ITO electrode is used as a cathode, it can be reduced electrochemically to a non-conductive. material. This cathodic reduction of ITO is irreversible under normal operating conditions: thereby fouling the electrode. To avoid the fouling of the transparent ITO electrode, photoactivated DEP relies on high frequency AC current to avoid such fouling. Since, DEP; force relies on dielectric constants that depend on the applied AC frequency, it is desirable to use low frequency AC current for the improved precision in the separation and manipulation of non-charged objects. However, low frequency AC current slowly deteriorates ITO resulting in loss of conductivity over time. It is desirable to replace the ITO with a transparent metal or metallic electrode, for example, a transparent gold electrode that is conductive cathodically or anodically.

EP whether optically activated or electrically activated can be used to separate or manipulate objects that have a charge such as DNA and cells that have a net charge on their surface. Typically, metal electrodes are used in a uniform or non-uniform electric field to provide the driving force to separate or manipulate objects. Electrically activated EP relies on metal electrodes to generate uniform or non-uniform electric fields, providing the driving force to separate or manipulate charged objects. Optically activated EP can rely on a transparent metal or metallic electrode that can permit a light beam, for example, a focused laser, to pass through the electrode and illuminate a photoconductive material adjacent to a non-transparent electrode, generating a non-uniform electric field and providing the driving force to separate or manipulate charged objects. It is desirable in either case to use an electrode material, for example, a transparent gold electrode that is conductive cathodically or anodically.

Whether metal or metallic, the electrodes can adsorb non-specifically biomolecules, such as proteins or nucleic acids in a biological sample, resulting in electrode fouling. This can occur whether the electrode is exposed to the biomolecules or polymers adjacent to the electrode are exposed to the biomolecules. It is desirable to add a surface modifier to the electrode to prevent non-specific adsorption of these biomolecules.

In the situation that a photoconductive material is covered with silicon nitride, a dielectric, it is desirable to replace the silicon nitride with a surface modified glass, or a polymer dielectric that can be surface modified to prevent non-specific adsorption of biological material in the liquid layer. It is also desirable to replace the silicon nitride with a semiconductive material that can be surface modified to prevent non-specific adsorption of biomolecules, for example, proteins from a biological sample.

In addition, it can be desirable to modify the surface of the glass or polymer in such a manner that arrays of specific ligands can be immobilized to specifically bind biomolecules and cells in the liquid biological sample layer.

### SUMMARY

In various embodiments, the present teachings can provide an optically activated manipulation chamber for biological material, including a liquid sample cavity including a first surface and a second surface, a transparent electrode positioned adjacent the first surface, wherein the transparent electrode includes a surface modifier to decrease the non-specific adsorption of the biological material to the transparent electrode, a photoconductive material positioned adjacent the second surface, and an electrode positioned adjacent the photoconductive material.

In various embodiments, the present teachings can provide a manipulation device for biological material, including a liquid sample cavity including a first surface and a second surface, a transparent electrode positioned adjacent the first surface, wherein the transparent electrode includes first a surface modifier to decrease the non-specific binding of the biological material to the transparent electrode, a transparent layer positioned adjacent the second surface, wherein the transparent layer includes second surface modifier to decrease the non-specific adsorption of the biological material to the transparent layer, a photoconductive material positioned adjacent the transparent layer, an electrode positioned adjacent the photoconductive material, a power source configured to provide an electrical potential difference between the transparent electrode and the electrode, and an illumination source for illuminating a portion of the photoconductive material with light, wherein the illuminated portion of the photoconductive material provides a region of manipulation between the transparent electrode and the electrode.

In various embodiments, the present teachings can provide a manipulation device for biological material, including a liquid sample cavity including a first surface and a second surface, a first electrode positioned adjacent the first surface, a second electrode positioned adjacent the second surface, and a power source configured to provide an electrical potential difference between the first electrode and the second electrode, wherein at least one of the first electrode and the second electrode includes a surface modifier to decrease the non-specific adsorption of the biological material to the at least one electrode.

In various embodiments, the present teachings can provide a method for dielectrophoretic cell manipulation, including providing a dielectrophoresis chamber, wherein at least a portion of the chamber is adapted for selective photo-activation, providing at least one cell for manipulation, and illuminating the portion of the chamber to provide a dielectrophoretic region adjacent to the cell, wherein the dielectrophoresis chamber is adapted to prevent non-specific adsorption of proteins of the cell.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description herein and appended claims.

### BRIEF DESCRIPTION OF THE FIGURES

FIGS. 1A-1C illustrate a cross-sectional view of three embodiments of an optically activated manipulation chamber in a dark state, according to the present teachings;

FIGS. 2A-2C illustrate a cross-sectional view of the three embodiments of the optically activated manipulation chamber illustrated in FIGS. 1A-1C in an illuminated state, according to the present teachings;

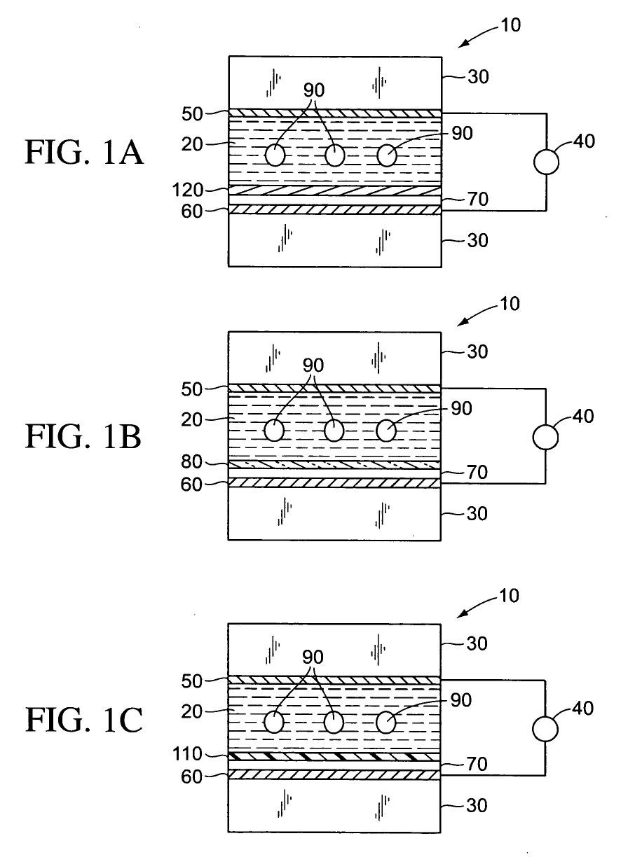

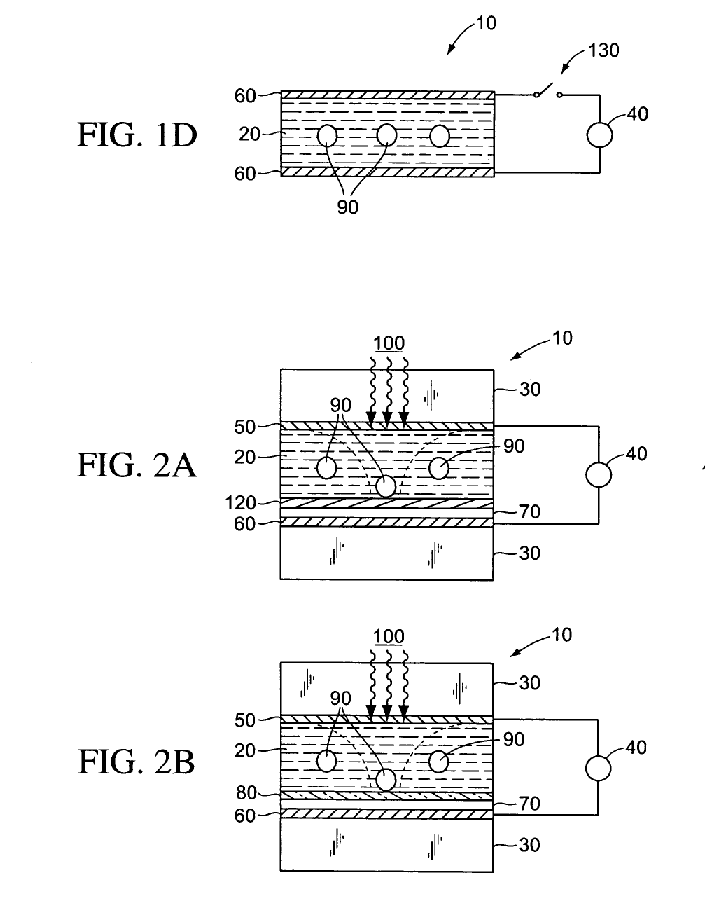

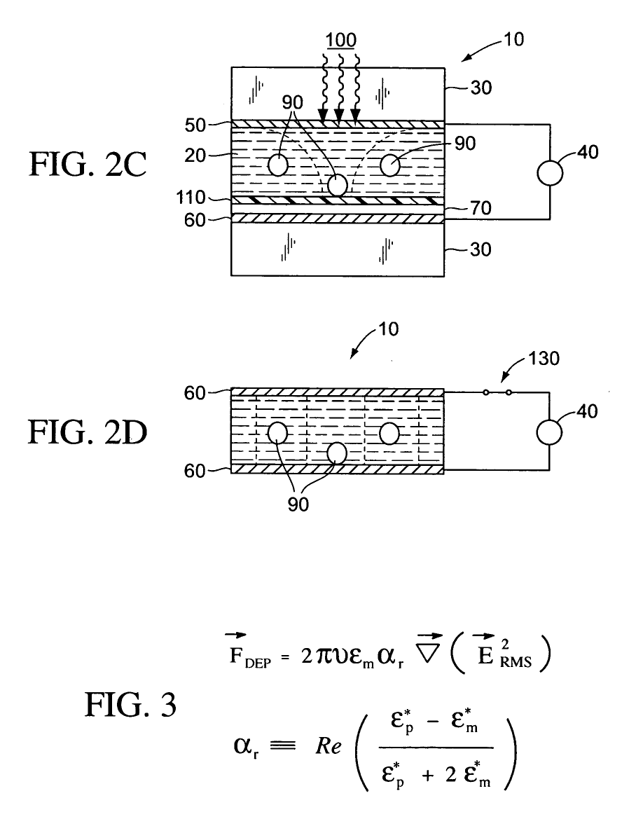

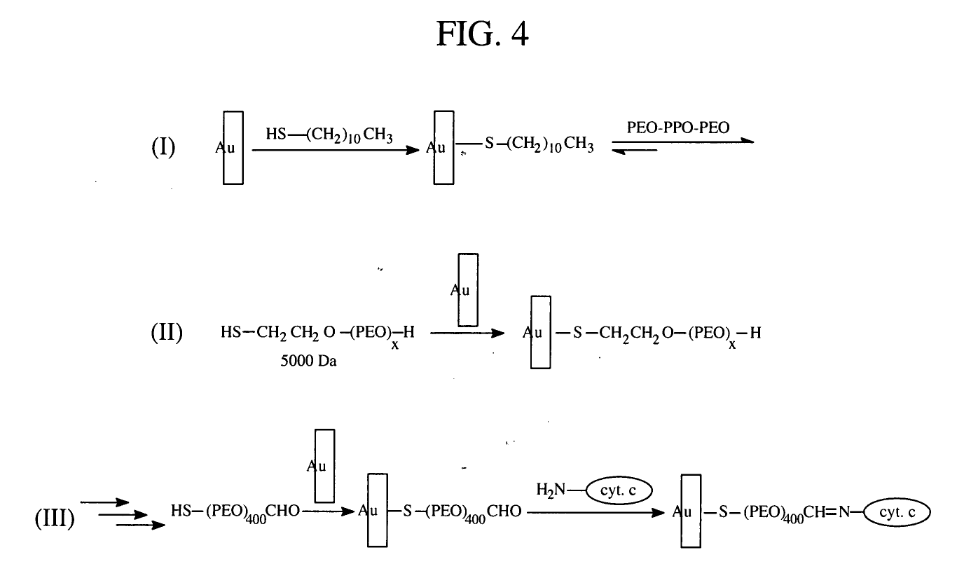

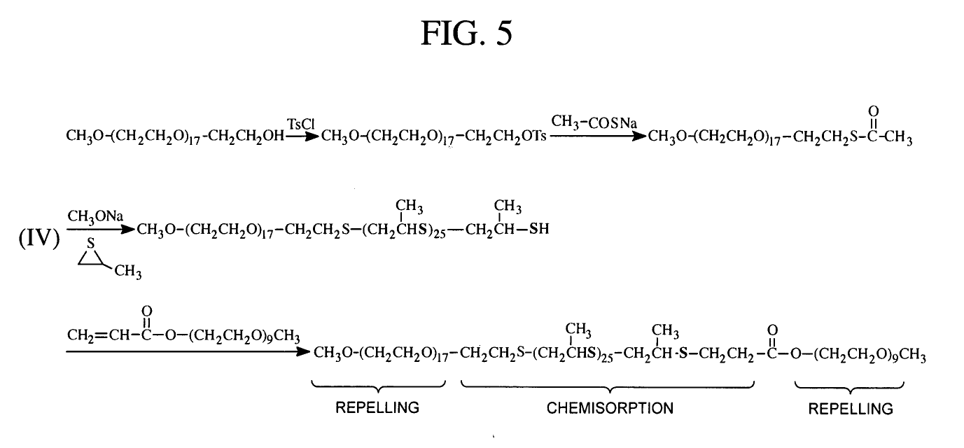

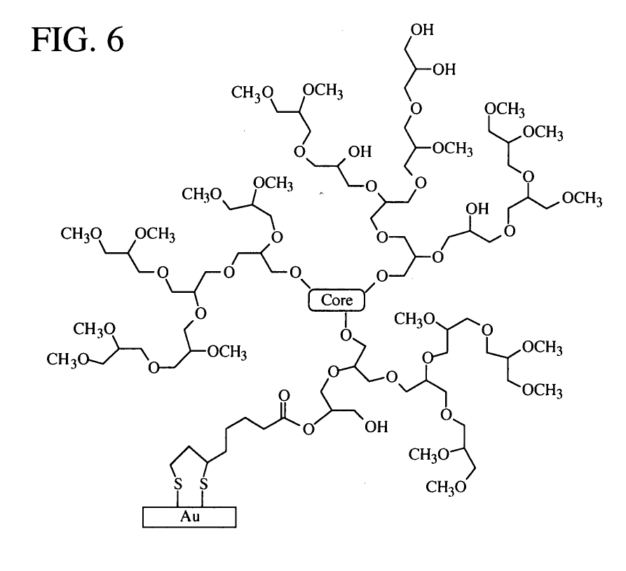

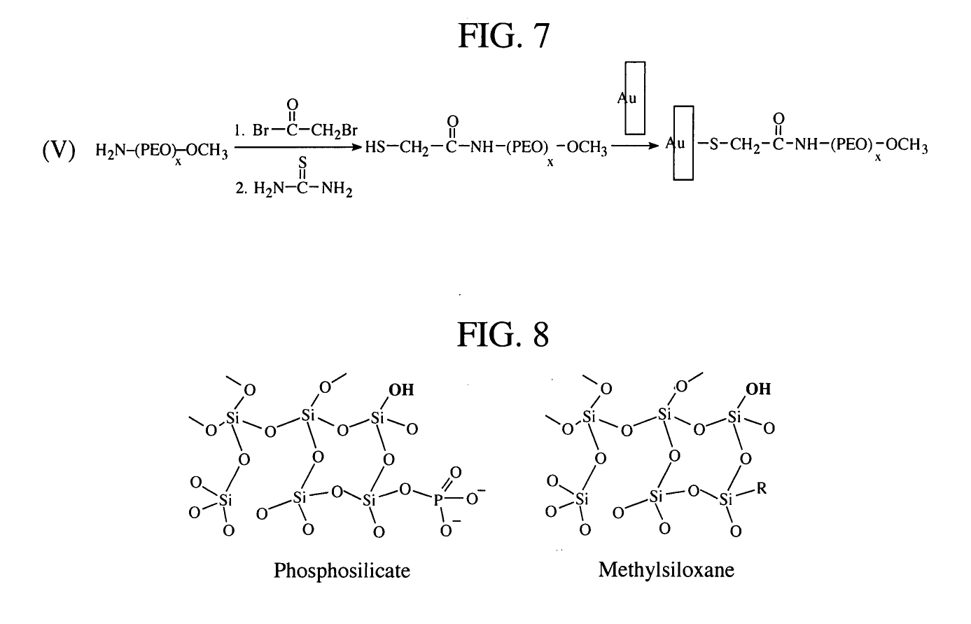

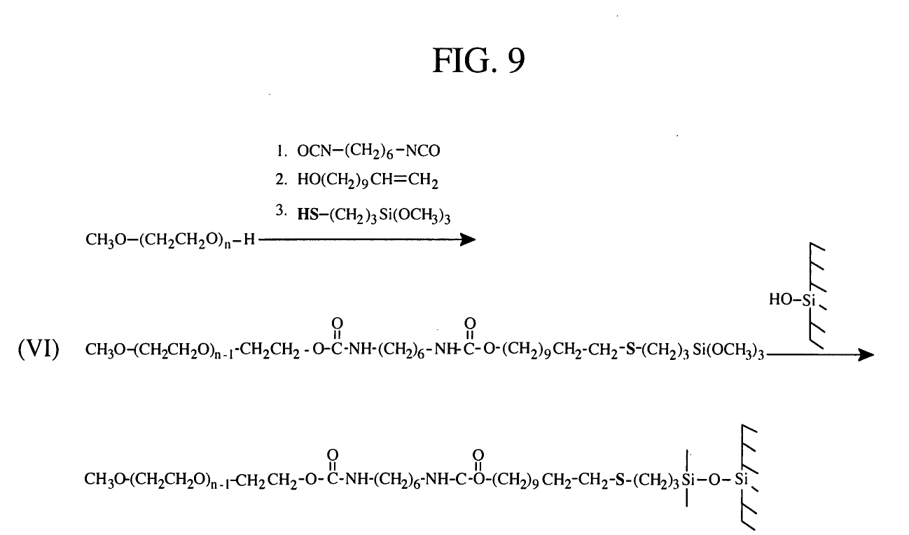

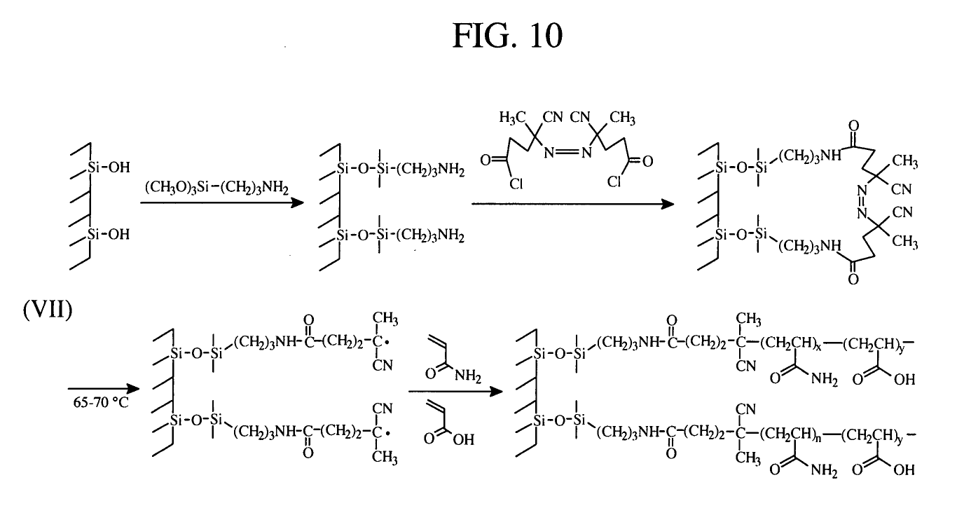

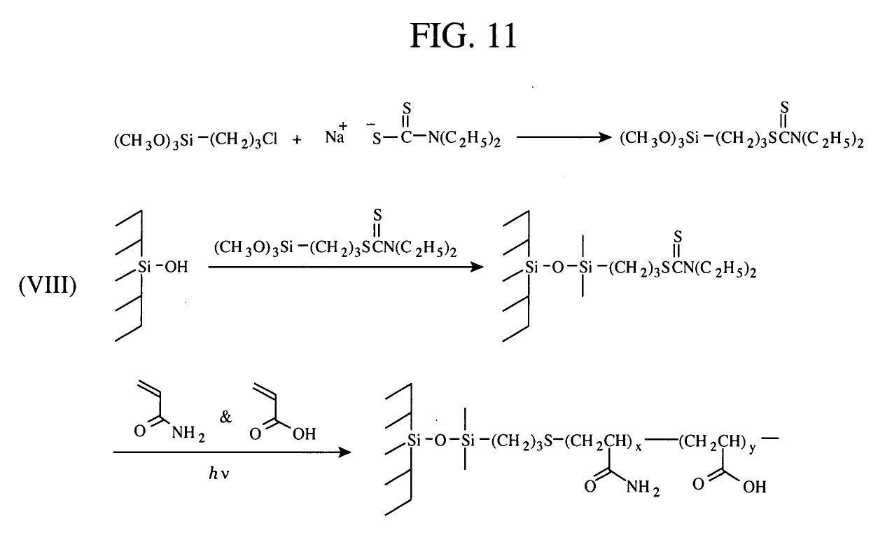

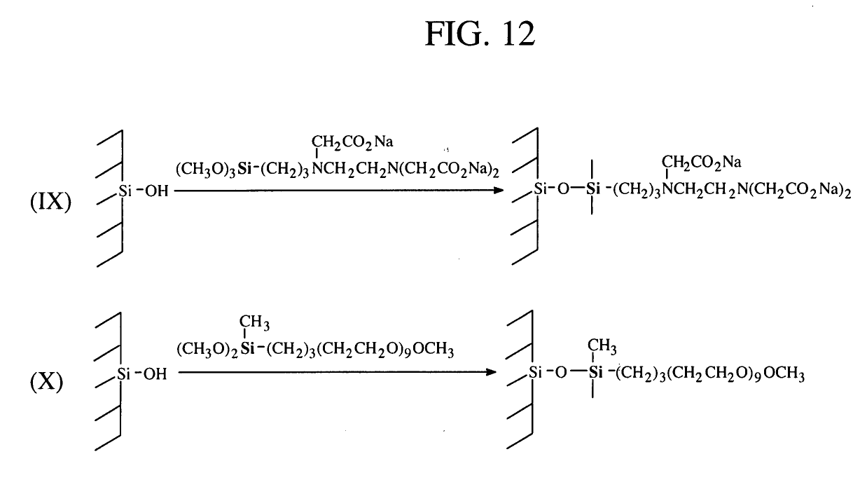

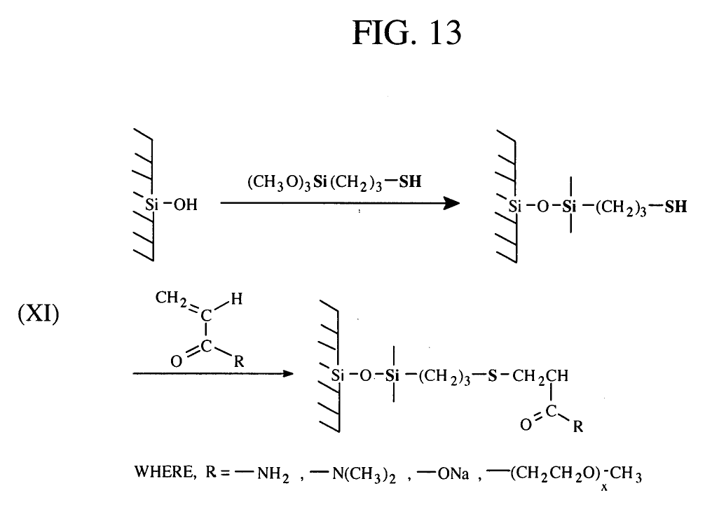

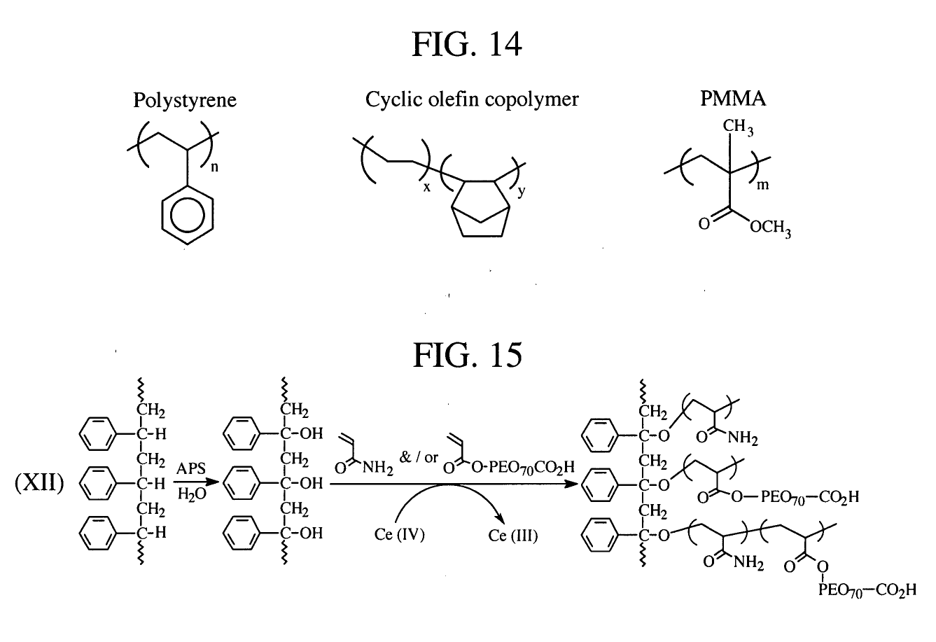

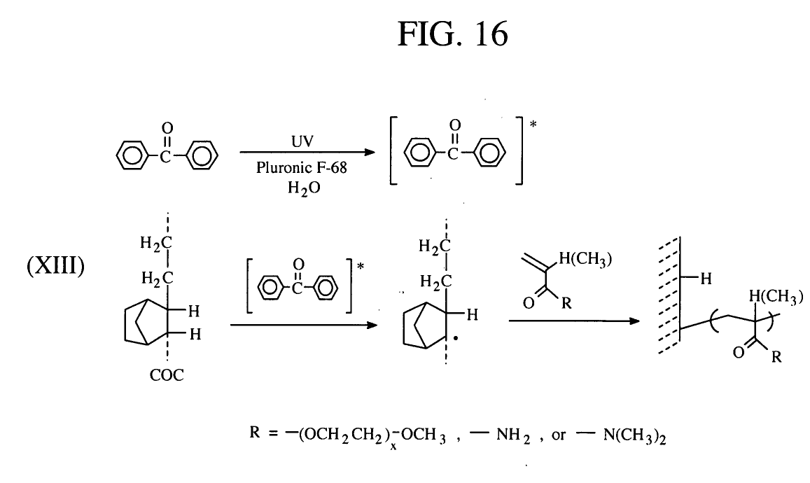

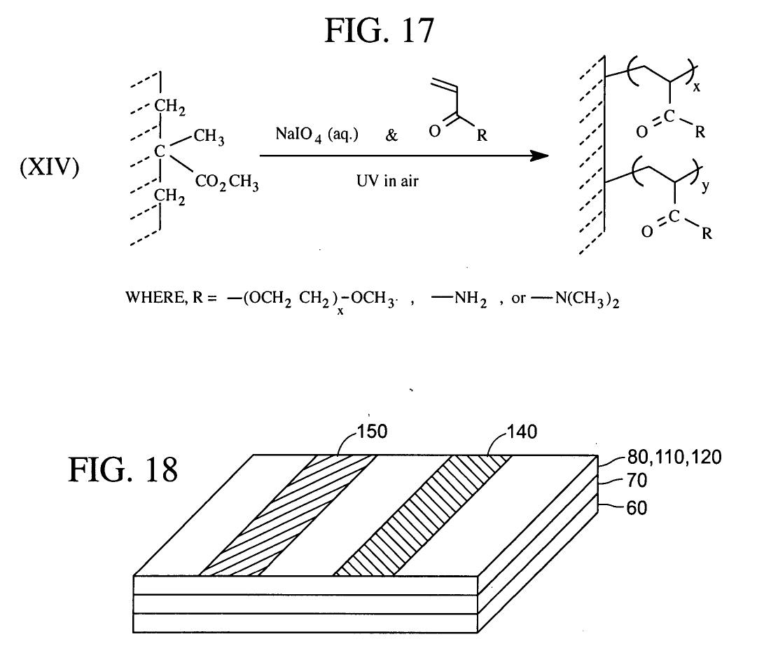

FIGS. 1D and 2D illustrate a cross-section view of an embodiment of a electrically activated manipulation chamber with an array of patterned electrodes, showing one set of electrodes with an open circuit and a close circuit, respectively, according to the present teachings;

FIG. 3 illustrates an analytical expression of DEP force.

FIGS. 4-7 illustrate several embodiments of surface modifiers for electrodes to reduce non-specific binding of proteins, according to the present teachings, including syntheses (I) to (V);

FIG. 8 illustrates examples of glass compounds;

FIGS. 9-13 illustrate several embodiments surface-modified glass to reduce non-specific binding of biological materials, according to the present teachings, including syntheses (VI) to (XI);

FIG. 14 illustrates examples of polymer layer compounds;

FIGS. 15-17 illustrate several embodiments surface-modified polymer layers to reduce non-specific binding of biological materials, according to the present teachings, including syntheses (XI) to (XIV); and

FIG. 18 illustrates a perspective view of a portion of an optically activated manipulation chamber with strips of cell-binding ligands, according to the present teachings.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

It is to be understood that both the foregoing general description and the following detailed description are exemplary and explanatory only, and are intended to provide an explanation of various embodiments of the present teachings.

### DESCRIPTION OF VARIOUS EMBODIMENTS

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Wherever possible, the same reference numbers will be used throughout the drawings to refer to the same or like parts.

The section headings used herein are for organizational purposes only, and are not to be construed as limiting the subject matter described. All documents cited in this application, including, but not limited to patents, patent applications, articles, books, and treatises, are expressly incorporated by reference in their entirety for any purpose. In the event that one or more of the incorporated literature and similar materials differs from or contradicts this application, including but not limited to defined terms, term usage, described techniques, or the like, this application controls.

The term “electrode” as used herein refers to the instrumentality used to provide electric current to the region of interest. An example of a metallic electrode is ITO and other compounds in the ITO family. Other metallic electrodes, for example metal oxides are described in M. Saif, et al., Proc. Intl. Conf. Vacuum Web Coating, 10^{th}, Fort Lauderdale, Fla., Nov. 10-12, 1996, pp. 286-300; C. G. Granqvist, et al., Appl. Phy. A: Solids and Surfaces, 1993, A57, 19-24; William R. Heineman, et al., Electroanalytical Chem., 1984, 13, 1-113. Examples of metal electrodes include gold, platinum, copper, aluminum, and other metals or alloys known in the electrical arts. Metal electrodes can result in transparent electrodes by sputter, spray, or vapor-deposit to form grids from 100 to 500 mesh of metal (or metals) on a transparent substrate as:known in the art (William R. Heineman, et al, Denki Kagaku oyobi Kogyo Butsuri Kagaku, 1982, 50, 142-8). For example, 10 nm Ni/Au can be used as a transparent electrode (Atsushi Motogaito, et al., Physica Status Solidi C: Conf. & Critical Review, 2003, 0(7), 147-150) can also be used. Optically transparent diamond electrodes, that exhibit super stability in aggressive solution environments without any micro-structural or morphological degradation (Greg M. Swain, et al., Abstract of Papers, 225^{th} ACS National Meeting, New Orlean, La., USA, Mar. 23-27, 2003; J. K. Zak, et al., Anal. Chem. 2001, 73 (5), 908-914). In the embodiments, where there optical activation of a photoconductive material through the surface of the electrode, it is desirable to have a transparent electrode. A transparent electrode permits at least a portion of illumination from a light source to reach the photoconductive material, even if the electrode is positioned between the illumination source and the photoconductive material. ITO is an example of a transparent electrode. Gold or platinum can be deposited in a thin layer on a transparent surface, such as glass. The layer of gold or platinum can be thick enough to provide conductivity and sufficiently thin, i.e., thinner than the wavelength of the illumination to permit the illumination to pass through the deposited layer of gold or platinum.

The term “photoconductive material” as used herein refers to a material that has different electrical conductivity properties in a dark state versus an illuminated state. For instance, the photoconductive material can be an insulator in a dark state and a conductor in an illuminated state. Examples of photoconductive materials include amorphous silicon. Other examples include amorphous selenium, polyferrocenylsilane, and other compounds known in the material science arts.

The term “surface modifier” as used herein refers to compounds capable of modifying the surface of an electrode to decrease non-specific adsorption of biomolecules in biological materials. Surface modifier compounds can include any material that can attach to the electrode, semiconductor, spin-on-glass, or polymer layer and provide hydrophilic characteristics to prevent non-specific adsorption of biomolecules. Examples of such materials include grafting of hydrophilic polymers, i.e. polymers with hydrophilic moieties, for example poly(ethylene glycol) or “PEO” of various molecular weights or polyacrylamide and its copolymers.

The term “illumination source” as used herein refers to any light source providing optical activation to complete the circuit providing a uniform or non-uniform electric field. An example of the illumination source is laser. However, an illumination source can be any light source with accompanying optical components that can provide focus for a beam of light that is on the scale of the biological object to be manipulated. For example, if a cell is the biological object to be manipulated, then the illumination source can provide a focused beam of light on the order of 1.0 to 10.0 microns, or the size of cell to be manipulated. Alternatively, if nucleic acid is the biological object to be manipulated, the illumination source can provide a focused beam of light on the order of 0.1 to 1.0 microns.

The term “power source” as used herein refers to AC or DC power supplies as known in the electrical arts. An AC or DC power supply can provide a uniform or a non-uniform electric field of variable frequency. The AC power supply can have a low frequency bias such that it approaches DC behavior.

The term “glass” and grammatical variations thereof as used herein refer to any glass layer that can be deposited proximate to the electrode, for example between the liquid layer and the photoconductive material. An example of glass that can be deposited is spin-on-glass (SOG). Commercially available examples of SOG include Accuglass® (Honeywell, Electrical Materials, Sunnyvale, Calif.), which includes T-03AS (thickness 1,040-3,070 Angstroms, dielectric constant at 1 MHz of 6-8, and refractive index at 633 nm of 1.43), P-5S (thickness 925-1,490 Angstroms, dielectric constant at 1 MHz of 4.7, and refractive index at 633 nm of 1.48), and T-12B (thickness 2,100-9,000 Angstroms, dielectric constant at 1 MHz of 3.2, and refractive index at 633 nm of 1.39).

The terms “polymer layer” as used herein refers to a material covering a surface uniformly or nonuniformly containing at least one polymer. The terms “polymer” refers to material resulting from polymerization. Polymers can include oligomers, homopolymers, and copolymers. Polymerization can be initiated thermally, photochemically, ionically, or by any other means known to those skilled in the art of polymer chemistry. According to various embodiments, the polymerization can be condensation (or step) polymerization, ring-opening polymerization, high energy electron-beam initiated polymerization, free-radical polymerization, including atomic-transfer radical addition (ATRA) polymerization, atomic-transfer radical polymerization (ATRP), reversible addition fragmentation chain transfer (RAFT) polymerization, or any other living free-radical polymerization.

The term “cell-binding ligands” as used herein refers to any material that can capture specific types of cells. Examples of such materials include lysosomes that capture Escherichia coli or Listeria monocytogens (T. Hung, et al. Enzyme and Microbial Tech., 2003, 33, 958-966), fibrinogen to bind platelets in whole blood (U.S. Pat. No. 5,854,005), polymers containing azlactone moiety capable of reacting with surface amino groups of a cell (U.S. Pat. No. 5,292,840), peptides or proteins that are specific for various surfaces of red blood cell membranes (WO 04/032970), reversible polyfunctional reagents binding to cells (WO 04/055213), phage ligands commercialized by Profos (Regensburg, Germany) to bind specifically to various bacteria, for example, Listeria spp, Listeria monocytogenes, salmonella spp, Escherichia coli 0157, and campylobacter spp (U.S. Pat. Appln. 2002/0127547A1), and ligands capable of capturing microbes (U.S. Pat. No. 6,780,602; WO 98/49557; H. Y. Kim, et al., IEEE Eng. Med. & Bio. Magazine, 2004, 122-129; H. Y. Mason, et al., Biosensors & Bioelectronics, 2003, 18, 521-527).

The term “non-specific adsorption of biological material” as used herein refers to indiscriminate adsorption, unintentional adsorption, or undesirable adsorption of biological material of interest to a random location, unknown location, or unwanted location on the electrode or proximate to the electrode.

The term “nucleic acid” as used herein refers to DNA, RNA, and variations of DNA and RNA, such as single strand DNA or double strand DNA, mRNA or iRNA.

In various embodiments, as illustrated in FIGS. 1A-1D, a manipulation chamber 10 for biological material 90 can include a circuit around liquid sample layer 20. FIGS. 1A-1C illustrate optically activated manipulation chambers in its dark state. Manipulation chamber 10 can include transparent substrates 30 to generally form a liquid sample cavity for liquid sample layer 20. A first side of the liquid sample cavity is formed by a transparent substrate 30 with a transparent electrode 50 and the second side is formed by a transparent substrate 30 with an electrode 60. In various alternative embodiments, the substrate adjacent to electrode 60 can be non-transparent and constructed of any material that can withstand the processing conditions for deposition of the photoconductive material. The transparent electrode 50 and electrode 60 are electrically coupled to power supply 40. In various embodiments, the transparent electrode can be gold or ITO, the power supply can be AC or DC, the electrode can be a thin aluminum electrode. In various embodiments, the AC current can have high frequency from 1 kHz to 10 MHz. In various embodiments, the AC current can have low frequency from less than 10 Hz to less than 1 kHz.

The circuit in FIGS. 1A-1C is closed by photoconductive material 70. In various embodiments, the photoconductive material 70 can be separated from the liquid sample layer 20 by a transparent material. The transparent material can be a polymer dielectric 110 (FIG. 1C), an insulating SOG 80 (FIG. 1B), a semiconductive SOG, a semiconductive transparent film 120 (FIG. 1A), or a silicon nitride film. FIGS. 2A-2C illustrate optically activated manipulation chambers 10 of FIGS. 1A-1C in an illuminated state by light 100. The light 100 can be focused to illuminate a portion of photoconductive material 70 closing the circuit between transparent electrode 50 and electrode 60. The closed circuit can generate an electric field between the activated portion of the photoconductive material 70 with adjacent electrode and the entire transparent electrode 50 opposite; field shown by the dashed line. The light 100 can be focused such that only the desired biological material 90, as shown the middle object, is manipulated.

FIG. 1D illustrates an electrically activated manipulation chamber is its open state. Switch 130 is in the open position prevent a circuit to form between electrodes 60 that are electrically coupled to power supply 40. The electrodes in the electrically activated manipulation chamber do not have to be the same. One of the electrodes can be configures as an array of individually controlled electrodes capable of providing a traveling wave to manipulate biological material. FIG. 2D illustrates the electrically activated manipulation chamber in a closed state with switch 130 in the closed position. The closed circuit can form an electric field between the electrodes 60; field shown by dashed lines. The biological material 90 in liquid sample layer 20 with a charge can be attracted to the electrode of opposite polarity.

In various embodiments, the selection of the surface modifiers can improve the DEP or EP performance. The surface of a metal or metallic electrode, polymer dielectric, an insulating SOG, a semiconductive SOG, a semiconductive transparent film, or a silicon nitride film can all be modified by surface modifiers to decrease non-specific adsorption of biological materials. FIGS. 4-7, 9-13, and 15-17 illustrate examples of surface modification for electrodes, SOG, and polymer layer. Although, one of each is used in each example, the surface modifiers and syntheses for modification can be interchangeable. The surface modifiers can be surface-grafted polymer or copolymer including monomer units such as, for example, ethylene oxide, propylene oxide, (meth)acrylamide, N-methyl(meth)acrylamide, N-ethyl(meth)acrylamide, N-iso-propyl(meth)acrylamide, N-n-propyl(meth)acrylamide, N,N-dimethyl(meth)acrylamide, N-ethyl-N-methyl(meth)acrylamide, N,N-diethyl(meth)acrylamide, N-vinylpyrrolidone, N-vinylacetamide, N-vinylformamides, N-methyl-N-vinylacetamide, 2-hydroxyethyl(meth)acrylate, 3-hydroxypropyl(methyl)acrylate, poly(ethyleneglycol)acrylate, poly(ethyleneglycol)(meth)acrylate, vinylmethyl ether, vinyl alcohol precursor, vinyloxazolidone, vinylmethyloxazolidone, N-(meth)acrylylcinamide, N-hydroxymethyl(meth)acrylamide, N-(3-hydroxypropyl)(methy)acrylamide, N-(meth)acryloxysuccinimide, N-(meth)acryloylmorpholine, N-acetyl(meth)acrylamide, N-amido(meth)acrylamide, N-acetamido(meth)acrylamide, N-tris(hydroxymethyl)methyl(meth)acrylamide, N-(methyl)acryloyltris(hydroxymethyl)methylamide, acryloylurea; and combinations thereof.

FIG. 4 illustrates surface modification by grafting poly(ethylene oxide) “PEO” and poly(ethylene glycol) “PEG” on a gold electrode. In synthesis (I), PEO is immobilized onto the surface through hydrophobic interaction between a PEO-PPO-PEO triblock copolymer and an anchored alkylthiol (P. Brandani, et al. Macromolecules, 2003, 36 (25), 6502-6509). In synthesis (II), ω-mercapto-PEG can be used to form a structure that is more stable than alkyl thiol (W. P. Wuelfing, et al., Abstract 215^{th} Natl. Mtg., Dallas, Mar. 29-Apr. 2 (1998)). In synthesis (III), PEO with Cytochrome C can be used (F. Kurisu, et al. Polym. Adv. Tech., 2003, 14 (1), 27-34). FIG. 5 illustrates surface modification by chemisorption of poly(propylene sulfide) on the electrode. In synthesis (IV), the surface modifier can have a central chemisorption section and repelling ends (J. P. Bearinger, et al., Nature Materials, 2003, 2, 259-264; A. Napoli, et al., Macromolecules, 2001, 34, 8913-8917). FIG. 6 illustrates a surface modifier with a core and dendromer ligands (C. Siegers, et al., Chem. Eur. J., 2004, 10, 2831-2838). FIG. 7 provides an example surface modification by grafting a methoxy-PEG as illustrated by synthesis (V), a two-step synthesis of α-methoxy-(ω-thioacetamido-PEG (N. Nagasshima, et al., Chem. Lett., 1996, (9), 731-732). In various embodiments, the surface modification can be performed with α-methoxy-ω-mercapto-PEG. In various embodiments, an acrylamide copolymer can replace PEO for modifications to the electrode or SOG.

In various embodiments, a glass, such as SOG can be deposited adjacent to the photoconductive material. FIG. 8 illustrates two examples of SOG, commercially available as Accuglass®, phosphosilicate (P-5S) and methylsiloxane (T-11). The methylsiloxane can behave as an insulator and the phosphosilicate SOG is more conductive. SOG can provide the benefits of thermal cure, planarization, high temperature stability (up to 900 degrees Celcius), crack resistance, good adhesion, and silanol for surface modification. FIG. 9 illustrates surface modification of SOG by synthesis (VI) including three-steps prior to surface attachment (S. Jo, et al., Biomaterials, 2000, 21, 605-616). FIG. 10 illustrates surface modification of SOG by synthesis (VII) with graft polymerization or graft copolymerization initiated thermally on the glass surface. (A. Yuyot, et al. Makromol. Chem, Macromol. Symp., 1993, 70/71, 265-274). FIG. 11 illustrates surface modification of SOG by synthesis (VI) with attachment and light mediated modification (U.S. Pat. No. 6,270,903). FIG. 12 illustrates surface modification of SOG using direct silylation by synthesis (IX) with one step reaction with negatively charged group and by synthesis (X) with one step reaction with neutrally charged group. FIG. 13 illustrates surface modification of SOG using Michael addition by synthesis (XI) with two steps forming a hydrolytically stable thiol linkage for a neutral or charged surface.

In various embodiments, a polymer layer, such as a polymer coating, can be deposited adjacent to the photoconductive material. FIG. 14 illustrates three examples of polymers that can be used for the polymer layer, such as polystyrene (PS), cyclic olefin copolymer (COC), and poly(methylmethacrylate) (PMMA). FIG. 15 illustrates surface modification of a polymer layer by Ce-mediated polymerization via hydroxide groups. Such modification can be applicable to polymers such as polycarbonates, polyolefins, COC, nylon, polyesters, etc. by syntheses like synthesis (XII) with acrylamide and PEO-acrylate and its derivatives to decrease passive adsorption of biomolecules (C. H. Bamford, et al. Polymer, 1996, 37, 4880-4889; C. H. Bamford, et al. Polymer, 1994, 35, 2844-2852; S. E. Shalaby, et al., Bull. NRC Egypt, 1993, 18, 189-202). FIG. 16 illustrates surface modification of a polymer layer by photo-initiated surface-grafting applicable to polymers such as PS, hydrogenated polystyrene, polypropylene, polydimethylsulfone, and PMMA by synthesis (XIII) with COC as example (T. Rohr, et al., Adv. Funct. Matl., 2003, 13, 264-267; T. B. Stachowiak, et al., Electrophoresis, 2003, 24, 3689-3693). FIG. 17 illustrates surface modification of a polymer layer by photo-initiated surface grafting by synthesis (XIV) with PMMA as example (Y. Ikada, et al., J. Appl. Polym. Sci., 1990, 41, 677-687; Y. Ikada, et al., J. Appl. Polym. Sci., 1993, 47, 417-424; T. Richey, et al., Biomaterials, 2000, 21, 1057-1065; S. Hu, et al., Anal. Chem., 2002, 74, 4117-4123; S. Hu, et al., Electrophoresis 2003, 24, 3679-3688).

In the case of transparent ITO electrode, the fouling is due to cathodic reduction rendering the ITO material non-conductive, i.e., disabling the electrode. The ITO electrode can only be used as an anode. This is an intrinsic characteristic of ITO. Applying high frequency AC current can help in prolonging the life span of the electrode, but eventually the ITO is reduced to a non-conductive material in time. In various embodiments, DEP with low frequency AC current or EP with DC current can be run with an electrode that can be conductive cathodically and anodically (i.e. it remains conductive when it is used as a cathode or an anode). Such electrodes benefit from surface modifications according the present teachings.

In various embodiments, the surface of silicon nitride can contain hydrophilic moieties such as, for example, hydroxyl, carboxyl, carboxylic, ammonium, poly(ethylene glycol), and combinations thereof through covalent bonding via a linker or passive adsorption on the surface.

In various embodiments, the surface modified electrode, SOG, semiconductor, polymer material, or silicon nitride can be further modified by cell-binding ligands to provide cell specific capture and manipulation. FIG. 18 illustrates a portion of an optically activated manipulation chamber with electrode 60, photoconductive material 70, and any one of semiconductor 120, SOG 80, or polymer layer 11 whose surface has be modified and portions of which are further combined with first cell-binding ligands region 140 and second cell-binding ligands region 150. For example, first cell-binding ligands can bind Escherichia coli and second cell-binding ligands can bind Listeria monocytogens. A liquid sample layer (not shown) with biological material, including Escherichia coli cells and Listeria monocytogens cells can be manipulated or moved over the surface of the manipulation chamber as described capturing the different cells in different regions. Then sequential solutions can be used to release the different cells to separate each type. In various embodiments, the cell-binding ligands regions can be arranged in an array. In various embodiments, photolithography can be used to designate the regions in the array for certain types of cell-binding ligands. In various embodiments, cell-binding ligands can be added to electrically activated manipulation chambers. In such manipulation chambers both surfaces in the liquid sample cavity can be non-transparent providing for cell-binding ligands to be added to both electrodes.

In various embodiments, the surface chemistry of a cell or particle and hence its permeability and εₚ can be selectively altered to provide specified sorting of certain cells. Certain cells can be selectively coated with a surface-active agent. This provides discrimination between different types of cells by modulating the permeability (dielectric constant) or surface net charge. Surface-active agents can selectively and specifically coat one type of cells but not others. For example, a non-ionic surface-active agent can be used to alter the permeability and/or dielectric constant such that DEP can provide cell sorting for otherwise charged cells. Alternatively, an ionic surface-active agent can be used to alter the permeability and/or dielectric constant such that EP can provide cell sorting for otherwise non-charged cells. Examples for non-ionic surface active agents are oligosaccharides for the capture of Bacillus Anthracis and Bordetella Pertusis. An example for ionic surface-active agents is positively charged hemin that binds to E. Coli 0157:H7 and Salmonella Typhi (U.S. Pat. Appln. 2004/0096910A1).

In various embodiments, the chamber for manipulation with optical activation can be incorporated as an integral part of an optical microscope. The chamber for manipulation can be an integral part of an optical microscope for sorting living cells, e.g. pathogen cells from mammalian cells, stem cells from muse skin cells (feeder cells). The chamber for manipulation of biological material could use the illumination source of the microscope and focus the light according to the present teachings. This could be done with conventional and confocal type of microscopes. The focusing lens of the microscope optics can be used to focus the light.

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to. “a polymer” includes two or more polymers. Furthermore, the use of the term “including”, as well as other forms, such as “includes” and “included”, is not limiting.

It will be apparent to those skilled in the art that various modifications and variations can be made to various embodiments described herein without departing from the spirit or scope of the present teachings. Thus, it is intended that the various embodiments described herein cover other modifications and variations within the scope of the appended claims and their equivalents.

What is claimed:

1. An optically activated manipulation chamber for biological material, the chamber comprising:
a liquid sample cavity comprising a first surface and a second surface;
a transparent electrode positioned adjacent the first surface, wherein the transparent electrode comprises a surface modifier to decrease the non-specific adsorption of the biological material to the transparent electrode;
a photoconductive material positioned adjacent the second surface; and
an electrode positioned adjacent the photoconductive material.

2. The manipulation chamber of claim 1, wherein an electric field between the transparent electrode and the electrode provides dielectrophoretic manipulation to substantially uncharged biological material.

3. The manipulation chamber of claim 2, wherein the biological material comprises a cell.

4. The manipulation chamber of claim 1, wherein the transparent electrode comprises gold.

5. The manipulation chamber of claim 4, wherein the surface modifier comprises a polymer.

6. The manipulation chamber of claim 5, wherein the polymer comprises a hydrophilic moiety with at least one moiety chosen from poly(ethylene oxide), acrylamide, hydroxyl, carboxyl, and ammonium.

7. The manipulation chamber of claim 1, further comprising a transparent layer adjacent to the second surface and the photoconductive material.

8. The manipulation chamber of claim 7, wherein the transparent layer comprises as least one of a semiconductive material, a spin-on-glass, and a polymer layer.

9. The manipulation chamber of claim 8, wherein the transparent layer comprises a surface modifier to decrease the non-specific adsorption of the biological material to the transparent layer.

10. The manipulation chamber of claim 9, wherein the surface modifiers provide an alkoxysilane moiety to attach to the spin-on-glass, wherein the spin-on-glass comprises a surface silanol group.

11. The manipulation chamber of claim 9, wherein the surface modifiers provide a hydrophilic moiety to decrease non-specific adsorption of the biological material.

12. The manipulation chamber of claim 11, wherein the hydrophilic moiety comprises at least one of poly(ethylene glycol), acrylamide, and carboxylic groups.

13. The manipulation chamber of claim 12, wherein the surface modifier is a polymer comprising at least one monomer unit chosen from ethylene oxide, propylene oxide, (meth)acrylamide, N-methyl(meth)acrylamide, N-ethyl(meth)acrylamide, N-iso-propyl(meth)acrylamide, N-n-propyl(meth)acrylamide, N,N-dimethyl(meth)acrylamide, N-ethyl-N-methyl(meth)acrylamide, N,N-diethyl(meth)acrylamide, N-vinylpyrrolidone, N-vinylacetamide, N-vinylformamides, N-methyl-N-vinylacetamide, 2-hydroxyethyl(meth)acrylate, 3-hydroxypropyl(methyl)acrylate, poly(ethyleneglycol)acrylate, poly(ethyleneglycol)(meth)acrylate, vinylmethyl ether, vinyl alcohol precursor, vinyloxazolidone, vinylmethyloxazolidone, N-(meth)acrylylcinamide, N-hydroxymethyl(meth)acrylamide, N-(3-hydroxypropyl)(methy)acrylamide, N-(meth)acryloxysuccinimide, N-(meth)acryloylmorpholine, N-acetyl(meth)acrylamide, N-amido(meth)acrylamide, N-acetamido(meth)acrylamide, N-tris(hydroxymethyl)methyl(meth)acrylamide, N-(methyl)acryloyltris(hydroxymethyl)methylamide, and acryloylurea.

14. The manipulation chamber of claim 8, wherein the transparent layer further comprises cell-binding ligands.

15. The manipulation chamber of claim 14, wherein the cell -binding ligands are positioned on the second surface to form an array.

16. The manipulation chamber of claim 1, wherein an electric field between the transparent electrode and the electrode provides electrophoretic manipulation to charged biological material.

17. The manipulation chamber of claim 16, wherein the charged biological material comprises nucleic acids.

18. A manipulation device for biological material, the device comprising:
a liquid sample cavity comprising a first surface and a second surface;
a transparent electrode positioned adjacent the first surface, wherein the transparent electrode comprises a first surface modifier to decrease the non-specific adsorption of the biological material to the transparent electrode;
a transparent layer positioned adjacent the second surface, wherein the transparent layer comprises a second surface modifier to decrease the non-specific adsorption of the biological material to the transparent layer;
a photoconductive material positioned adjacent the transparent layer;
an electrode positioned adjacent the photoconductive material;
a power source configured to provide an electrical potential difference between the transparent electrode and the electrode; and
an illumination source for illuminating a portion of the photoconductive material with light,
wherein the illuminated portion of the photoconductive material provides a region of manipulation between the transparent electrode and the electrode.

19. The manipulation device of claim 18, wherein the region of manipulation between the transparent electrode and the electrode provides dielectrophoretic manipulation to substantially uncharged biological material.

20. The manipulation device of claim 18, wherein the power source provides AC current.

21. The manipulation device of claim 20, wherein the AC current has high frequency from 1 kHz to 10 MHz.

22. The manipulation device of claim 20, wherein the AC current has low frequency from less than 10 Hz to less than 1 kHz.

23. The manipulation device of claim 18, wherein the biological material comprises a cell.

24. The manipulation device of claim 23, wherein the manipulation device is incorporated into an optical microscope.

25. The manipulation device of claim 18, wherein the transparent electrode comprises gold.

26. The manipulation device of claim 18, wherein the first surface modifier and the second surface modifier is the same.

27. The manipulation device of claim 18, wherein an electric field between the transparent electrode and the electrode provides electrophoretic manipulation to charged biological material.

28. The manipulation chamber of claim 27, wherein the charged biological material comprises nucleic acids.

29. A manipulation device for biological material, device comprising:
a liquid sample cavity comprising a first surface and a second surface;
a first electrode positioned adjacent the first surface;
a second electrode positioned adjacent the second surface; and
a power source configured to provide an electrical potential difference between the first electrode and the second electrode; and
wherein at least one of the first electrode and the second electrode comprises a surface modifier to decrease the non-specific adsorption of the biological material to the at least one electrode.

30. The manipulation device of claim 29, wherein the surface modifier comprises a polymer.

31. The manipulation device of claim 30, wherein the polymer comprises a hydrophilic moiety with at least one moiety chosen from poly(ethylene oxide), acrylamide, hydroxyl, carboxyl, and ammonium.

32. The manipulation device of claim 31, wherein the polymer comprises a surface modifier is a polymer comprising at least one monomer unit chosen from ethylene oxide, propylene oxide, (meth)acrylamide, N-methyl(meth)acrylamide, N-ethyl(meth)acrylamide, N-iso-propyl(meth)acrylamide, N-n-propyl(meth)acrylamide, N,N-dimethyl(meth)acrylamide, N-ethyl-N-methyl(meth)acrylamide, N,N-diethyl(meth)acrylamide, N-vinylpyrrolidone, N-vinylacetamide, N-vinylformamides, N-methyl-N-vinylacetamide, 2-hydroxyethyl(meth)acrylate, 3-hydroxypropyl(methyl)acrylate, poly(ethyleneglycol)acrylate, poly(ethyleneglycol)(meth)acrylate, vinylmethyl ether, vinyl alcohol precursor, vinyloxazolidone, vinylmethyloxazolidone, N-(meth)acrylylcinamide, N-hydroxymethyl(meth)acrylamide, N-(3-hydroxypropyl)(methy)acrylamide, N-(meth)acryloxysuccinimide, N-(meth)acryloylmorpholine, N-acetyl(meth)acrylamide, N-amido(meth)acrylamide, N-acetamido(meth)acrylamide, N-tris(hydroxymethyl)methyl(meth)acrylamide, N-(methyl)acryloyltris(hydroxymethyl)methylamide, and acryloylurea.

33. The manipulation device of claim 29, wherein the at least one electrode further comprises cell-binding ligands.

34. The manipulation device of claim 33, wherein the cell-binding ligands are positioned on the at least one electrode to form an array.

35. The manipulation device of claim 29, wherein the electric potential difference provides dielectrophoretic manipulation to substantially uncharged biological material.

36. The manipulation device of claim 29, wherein electric potential difference provides electrophoretic manipulation to charged biological material.

37. A method for dielectrophoretic cell manipulation, comprising:
providing a dielectrophoresis chamber, wherein at least a portion of the chamber is adapted for selective photo-activation;
providing at least one cell for manipulation; and
illuminating the portion of the chamber to provide a dielectrophoretic region adjacent to the cell;
wherein the dielectrophoresis chamber is adapted to prevent non-specific adsorption of proteins of the cell.

38. The method of claim 37, further comprising treating the cell with a surface active agent.

39. The method of claim 37, further comprising capturing the cell with cell-binding ligands.

40. The method of claim 39, further comprising eluting the cell with a release liquid.
